(19) United States
(12) Patent Application Publication
(10) Pub. No.: US 2014/0357958 A1
(43) Pub. Date: Dec. 4, 2014

(54) CANNULA TOP AND SYSTEM

(71) Applicant: Surgical Innovations Limited, Leeds, Yorkshire (GB)

(72) Inventors: John Eltoft, Leeds (GB); Vhairi Maxwell, Leeds (GB); Michael White, Leeds (GB); Ross Richardson, Auchterarder (GB)

(21) Appl. No.: 14/362,226

(22) PCT Filed: Nov. 21, 2012
(86) PCT No.: PCT/GB2012/052885

§ 371 Date: Jun. 2, 2014

(30) Foreign Application Priority Data
Dec. 2, 2011 (GB) 1120736.2
Sep. 4, 2012 (GB) 1215745.9

(51) Int. Cl. A61B 17/34

(52) U.S. Cl. CPC A61B 17/3498; A61B 17/3423; A61B 17/3439; A61B 2017/3441; A61B 2017/00477
USPC 600/204

(57) ABSTRACT

A cannula top for enclosing a surgical seal which comprises a base member having a through bore which defines a receptacle for the surgical seal and a lid member having a through bore. The lid member is movable between a first position in which the surgical seal can be inserted into and removed from the receptacle, and a second position in which the surgical seal is engaged by the lid member and retained in the receptacle by the lid member. The cannula top further comprises a connecting member which connects the lid member to the base member in both the first position and the second position.

The present application relates to a cannula top, and in particular to a cannula top which can enclose a surgical seal or valve. The present invention also relates to a system comprising such a cannula top and a surgical seal.

Cannulas are used during minimally invasive surgery to provide access to a body cavity. Typically, a body cavity will be inflated during the minimally invasive surgical procedure. For example, during a laparoscopic procedure, the abdomen may be insufflated. When the body cavity is insufflated, it is necessary to provide a valve or seal at all points of entry into the body cavity.

The YelloPort+plus® system commercially available from Surgical Innovations Limited, Leeds, UK provides a system of cannula tops which enclose a replaceable valve or seal. Typically, the cannula top has two parts. A first part is connected to the cannula itself and has a through bore which defines a receptacle for the surgical seal. The surgical seal is formed from a relatively soft and flexible material, such as silicone, and is inserted into the receptacle and secured in place by a second part. The second part also has a through bore and is screwed in place onto the first part, so that the through bore allows access to the seal. The screw fit sandwiches the surgical seal between the first and second part, providing a seal into the body cavity accessed through the cannula.

There is a disadvantage that because the cannula top has two parts, one part may become separated from the other during sterilisation and/or decontamination after use. This can cause problems with inventory control. It would be desirable to provide a cannula top in which inventory control is improved.

A cannula top is typically designed to be used with a seal corresponding to a particular diameter of surgical instrument shaft. Systems have been proposed which include adaptors allowing use with more than one diameter of surgical instrument shaft. For example, WO-2008/023200 discusses a cannula sealing apparatus in which a single use housing is attached via a bayonet connection to enclose and engage a seal. The cannula housing includes two pivoting members which can be positioned above the enclosed seal to change diameter of an opening for a surgical instrument. This allows use with more than one diameter of surgical instrument but the resulting cannula top still has separate parts which engage and retain the seal. The cannula top of WO-2008/023200 is single use so the disadvantages of inventory control during sterilisation/decontamination do not occur.

While other forms of adaptor system have also been proposed, for example EP-1716813A discusses a diameter changing adaptor with a tether that is mountable on cannula sleeve, the seal is still retained by two separate pieces, giving problems with inventory control.

A further disadvantage is that the separate elements forming the cannula top may be reasonably small, so that one part may be dropped during assembly. A dropped part cannot be used in surgery without sterilisation and/or decontamination.

Accordingly, the present invention provides a cannula top in which the lid member is connected to the base member in both the first position and the second position. This reduces the risk of the lid member being separated from the base member in the first position. It is advantageous during sterilisation or decontamination of the base member and lid member because the lid member and base member need to be in the first position for sterilisation or decontamination. The risk of one of the lid member or base member being lost or separated during autoclaving is reduced because they remain connected in the first position.

A further advantage of the lid member being connected to the base member in both the first and second position is that it enables use of cannula tops with different diameter through bores but similar external dimensions without risk of confusing the lid members and base members of different diameter cannula tops in the operating theatre. For example, in some embodiments the through bore may have a 5 mm diameter or a 3 mm diameter, with the same external dimensions for both a 5 mm and a 3 mm through bore. In use in the operating theatre, the difference between the 3 mm and 5 mm through bore is relatively small and the similar external dimensions may mean that a 3 mm lid is mistakenly combined with a 5 mm base or vice versa. Time may also be wasted in the operating theatre matching the correct size base and lid members. The connection between the lid member and base member in both the first position and the second position reduces these problems.

According to a first aspect of the invention, there is provided a cannula top for enclosing a surgical seal, wherein the cannula top comprises:
a base member having a longitudinal axis and a through bore which defines a receptacle for the surgical seal; and
a lid member having a through bore and movable between a first position and a second position, wherein in the first position the surgical seal can be inserted into the receptacle, and in the second position the surgical seal is engaged by the lid member and retained in the receptacle; wherein
the lid member is connected to the base member in both the first position and the second position.

According to another aspect of the invention, there is provided a cannula top for enclosing a surgical seal, wherein the cannula top comprises:
a base member having a through bore which defines a receptacle for the surgical seal;
a lid member having a through bore, wherein the lid member is movable between a first position in which the surgical seal can be inserted into and removed from the receptacle, and a second position in which the surgical seal is engaged by the lid member and retained in the receptacle by the lid member; and
a connecting member which connects the lid member to the base member in both the first position and the second position.

The surgical seal preferably has rotational symmetry around an axis which is coincident with the longitudinal axis of the through bore when it is inserted into the receptacle of the base member. The surgical seal preferably comprises a portion comprising a continuous rim that may be engaged by the lid member to form a seal. The surgical seal can then provide air-tight access to a cannula when the lid member is in the first position. The through bore in the base member and the through bore in the lid member provide a channel for inserting an instrument through the seal to access the cannula. The connecting member may be flexible but is preferably substantially rigid.

In one embodiment, the lid member may be permanently connected to the base member.

In one embodiment the base member and the lid member each define respective threads and the lid member is retained in the second position by the engagement of the thread of the base member with the thread of the lid member. This provides a reliable connection between the lid member and the base member in the second position. The thread may have any suitable profile, for example, it may be a helical thread. In alternate embodiments other means of connection may be used, for example a bayonet or snap-fit connection.

The cannula top is preferably rotatably connected to the connecting member for rotation relative to the connecting member about the axis of the through bore of the lid member. When the through bore is threaded, the axis of the thread is preferably coaxial with the axis of the through bore of the lid member. In use, this allows the lid member to be rotated relative to the base member, for example to engage and disengage a threaded or bayonet connection.

The lid member is preferably connected to the connecting member for translation relative to connecting member in the direction of the axis of the through bore defined by the lid member.

Preferably, the lid member defines an outer surface which comprises a cylindrical portion and the connecting member engages the lid member via a ring which surrounds the cylindrical portion. This provides a rotatable construction with a simple construction.

The cylindrical portion preferably has a length which is greater than the axial thickness of the ring. The axial thickness is the dimension perpendicular to the plane defined by the ring. This construction allows for both rotation and translation of the lid member relative to the ring on the connecting member. A further advantage is that the translation assists the sterilisation and/or decontamination process after use.

Preferably, the connecting member is pivotally connected to the base member. This allows the lid member to be moved away from the through bore in the base member when the lid member is in the first position, improving access to the receptacle defined by the base member.

Preferably, the lid member is connected to the base member by a joint having at least two degrees of freedom. This allows sufficient freedom of movement for a seal to be installed and removed, while allowing a simple construction.

Preferably, the lid member is connected to the base member via a joint which allows rotation about an axis which is substantially perpendicular to the longitudinal axis and translation in the direction perpendicular to the axis.

In one embodiment, the joint is provided by a linear track and a pin engaging the track. For example, either the base member or the lid member may define a linear track and the lid member is attached to the base member by a pin engaging the track. In such a construction it is preferred that the lid member defines a linear track. This means of connection provides two degrees of freedom between the lid member and the base member, while keeping them connected at all times. The pin can be slid along the linear track to provide translation and relative rotation can take place about the axis of the pin.

The applicant has identified that in some circumstances using a threaded connection the seal into the body cavity may not be completely effective. For example, the seal may not be completely air tight if the instrument is moved relative to the seal during surgery.

While investigating the effectiveness of the prior art YelloPort+plus® seal, the applicant has been found that the screw connection between the first and second parts of the cannula top can introduce distortions into the seal. When the seal is held within the cannula top of the prior art YelloPort+plus® system, the screw connection of the first part to the second part results in shear forces being applied to the seal. These shear forces may introduce distortions into the seal, which can create points of weakness where the seal is less effective. For example, the rim of the seal which is gripped between the first part and second part may distort in the radial direction in response to the shear forces.

To address this disadvantage, when a threaded connection is used between the lid member and the base member, it is preferable that the lid member further comprises a rotating member which is retained within the lid member and mounted for rotation relative to the lid member about the axis of the thread of the lid member. This rotating member can then engage an inserted seal in use and prevent the transfer of shear forces to the seal. For example, the coefficient of friction between the seal and the rotating member may be greater than the coefficient of friction between the rotating member and the lid member. Thus, the relative rotation as the threaded connection is tightened occurs between the rotating member and the lid member. Only the longitudinal force is transferred to the seal, not the shearing forces from rotation. In some embodiments, the rotatable member may also be mounted for translation relative to the lid member along the axis of the thread of the lid member.

In an alternative embodiment, a snap fit connection rather than a threaded connection may be used, for example the lid member may be retained in the second position by a snap-fit connection to the base member. The snap-fit resulting from a longitudinally applied force means that there is no rotational component to the force applied to the seal and therefore no shear forces applied to a seal within the cannula top. The distortions of the seal are therefore avoided and the seal will function more reliably.

When a snap-fit connection is used, the base member may further comprise at least one retaining member comprising a first formation and the lid member further comprises a second formation, wherein in the second position the first formation engages the second formation to retain the lid member in the second position. The combination of the first and second formation provides the snap-fit connection in the second position. The first and second formations may have any suitable form. For example, the first formation may be a projection and the second formation may be a corresponding recess, and vice versa.

Preferably, the retaining member is movable in the direction towards longitudinal axis to disengage the first formation from the second formation. This allows the snap-fit connection to be released manually enabling movement of the lid member out of the second position so that an enclosed seal can be accessed and removed, for example for sterilisation/decontamination and/or replacement of the seal. Preferably, the retaining arm is resilient. In that case an applied force can deforms the retaining arm resiliently in the direction towards the longitudinal axis, the retaining arm will then return to its previous position when the applied force is removed.

Preferably, the retaining member is connected to the base member for rotation between a locking position and an open position. In the locking position the retaining member is substantially parallel to the longitudinal axis and the first formation is positioned to engage the second formation. In the open position the retaining member extends at least partially outward from the longitudinal axis. In this embodiment, the retaining member can rotate to an open position. In the open position access to the seal within the receptacle is improved, which enables easier removal of the seal after use. A further advantage is that when the retaining member is in the open position, access to the through bore and receptacle is improved for sterilisation/decontamination.

In some embodiments, the cannula top further comprises attachment means for releasable attachment to the end of a cannula. The attachment means can be any mechanism suitable for connecting the cannula top to a cannula. Examples include screw thread connections, snap-fit connections or use of additional components such as a locking collar or mechanism.

In other embodiments, the base member may form an integral part of a cannula. In that case, the cannula extends distally from the end of the base member furthest from the lid member.

According to another aspect of the present invention, there is provided a system comprising a cannula top as described above (with or without the optional features also described) and a surgical seal.

In a further aspect of the invention, the snap-fit connection may be provided without the connecting member. This provides the benefit of reducing shear forces applied to the seal in use.

In another aspect, the rotating member within the lid member may be provided without the connecting member. For example a prior art YelloPort+plus® system may be adapted to include the rotating member in its lid member. This provides the benefit of reducing shear forces applied to the seal in use.

Embodiments of the invention will now be described with reference to the accompanying drawings by way of example only, in which:

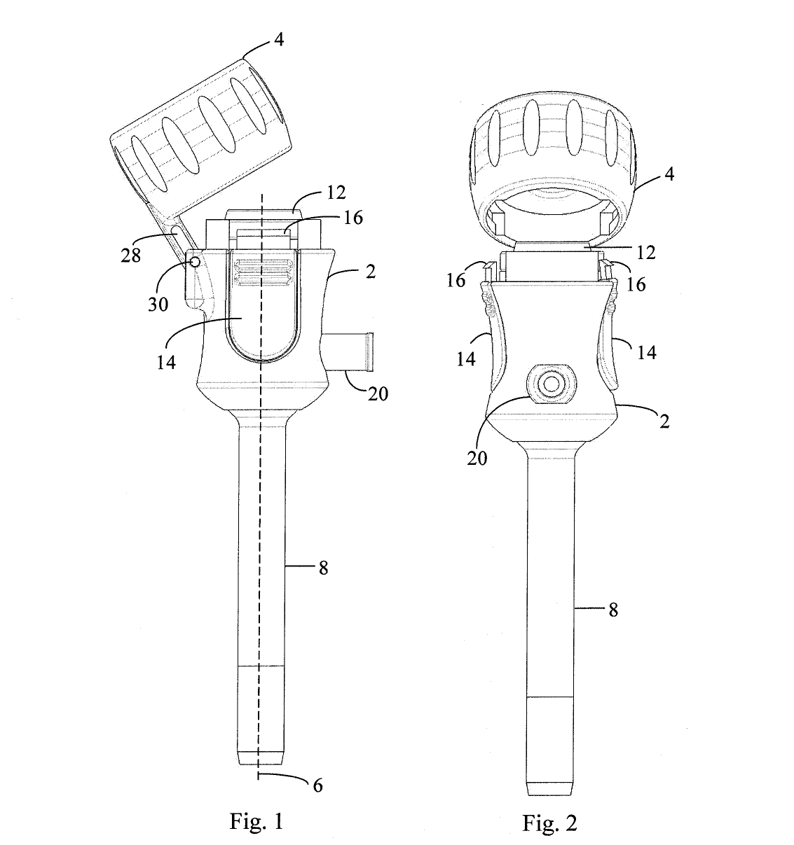

FIG. 1 depicts a side view of a cannula top according to an embodiment of the present invention;

FIG. 2 depicts a side view of the cannula top of FIG. 1 rotated through 90°;

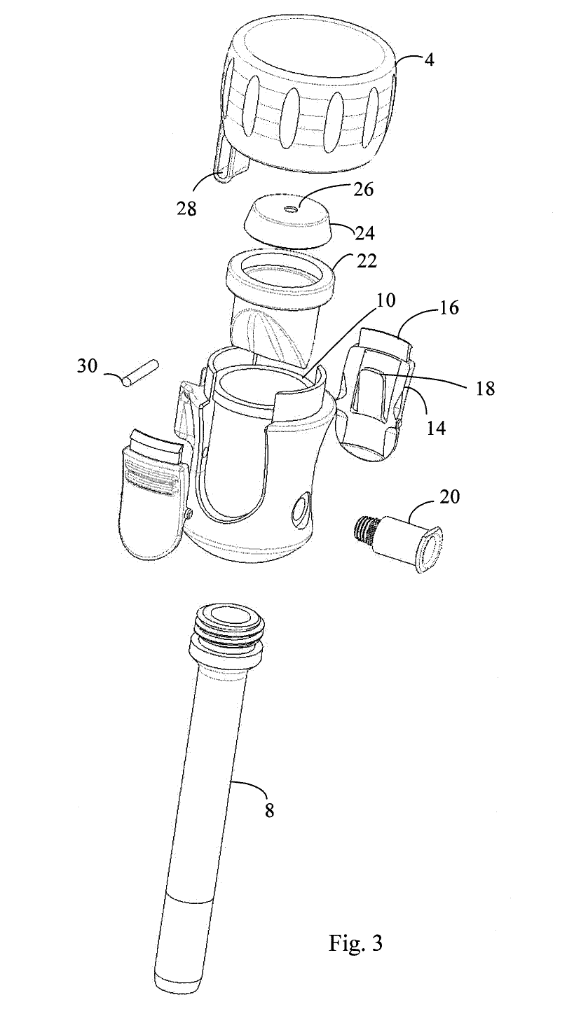

FIG. 3 depicts an exploded diagram of the components of the cannula top of FIG. 1;

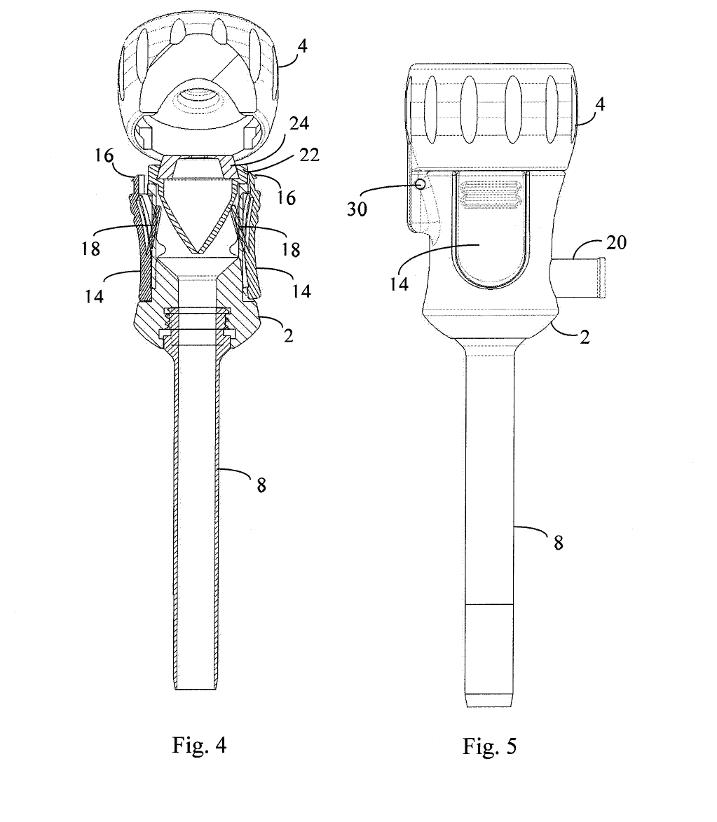

FIG. 4 depicts a partial cross-section through the cannula top in the position shown in FIG. 1;

FIG. 5 depicts a side view of a cannula top, when the cannula top is in a second position enclosing a surgical seal;

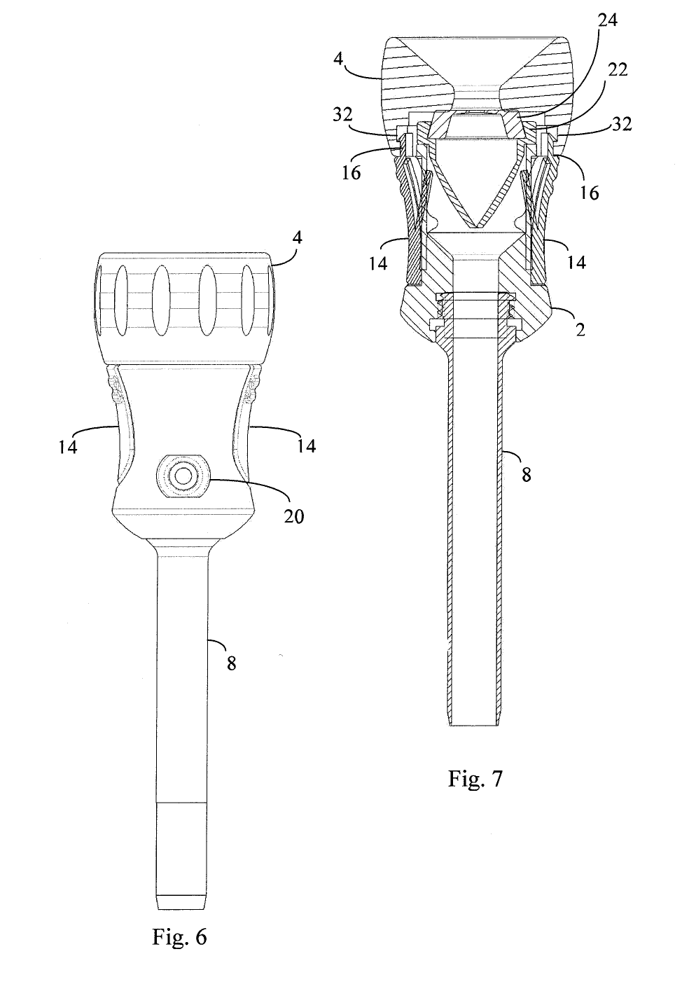

FIG. 6 depicts a view from the rear of FIG. 6, i.e. a view rotated through 90° compared with FIG. 5;

FIG. 7 depicts a cross-section through the cannula top of FIGS. 5 and 6;

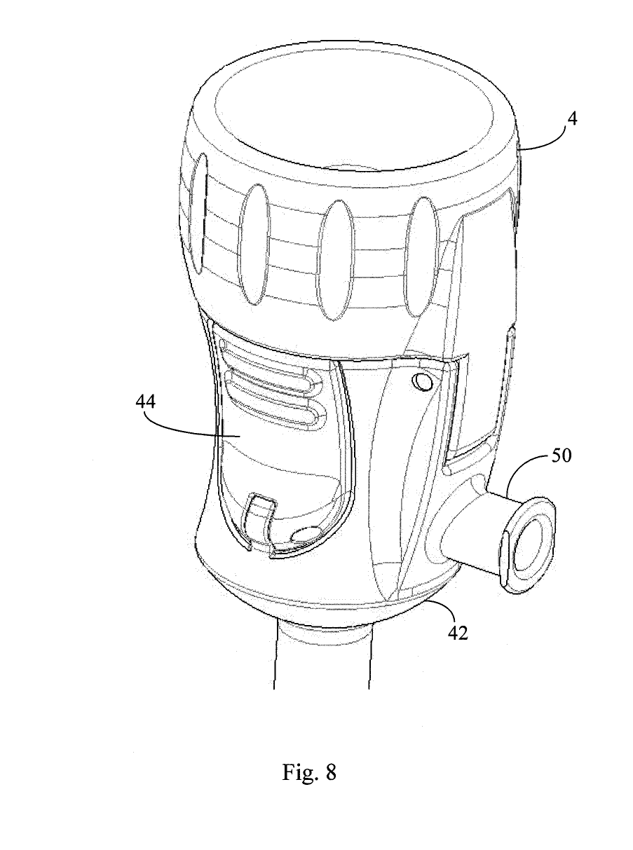

FIG. 8 depicts a perspective view of a cannula top according to a second embodiment of the invention;

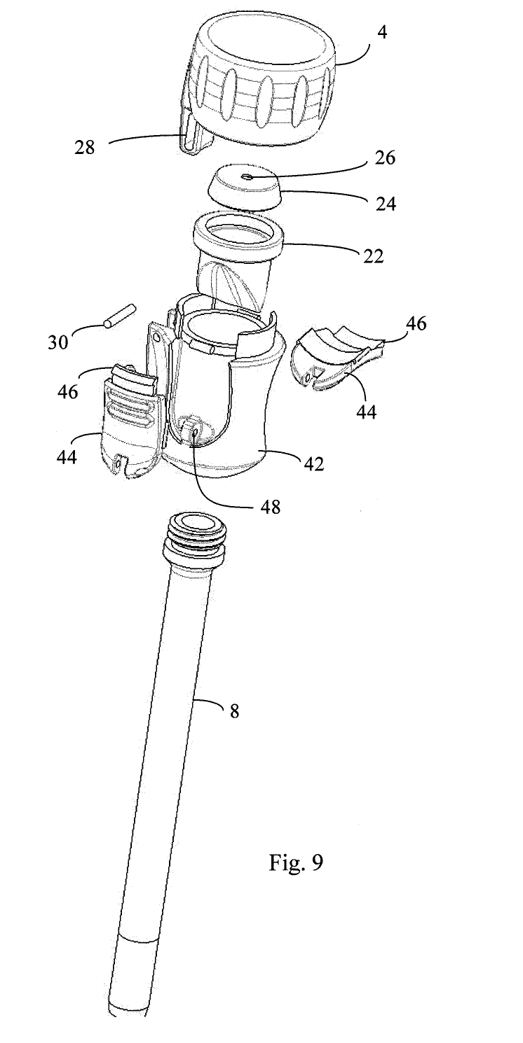

FIG. 9 depicts an exploded view of the cannula top of FIG. 8;

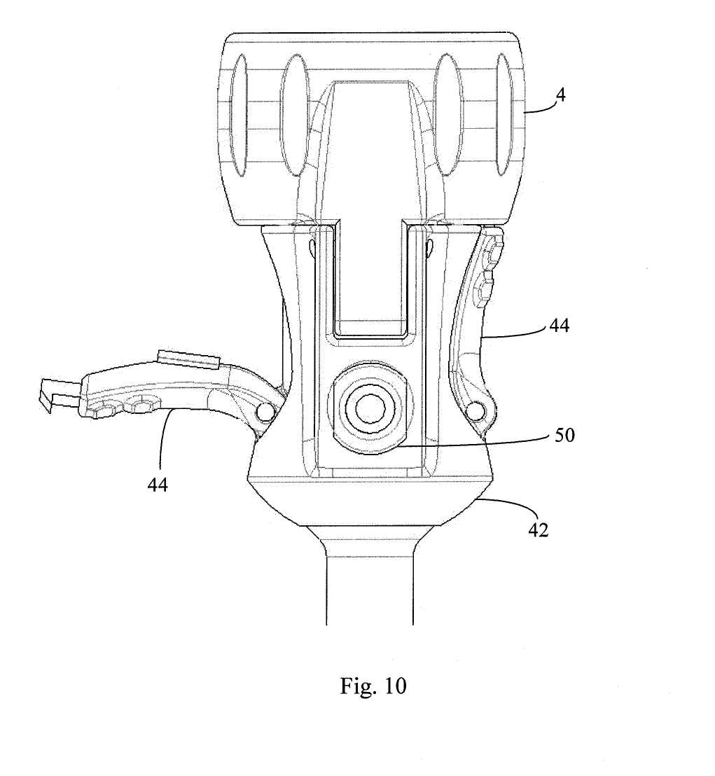

FIG. 10 depicts a side view of the cannula top of FIG. 8 with one of the retaining arms in an open position and the other of the retaining arms in the closed position;

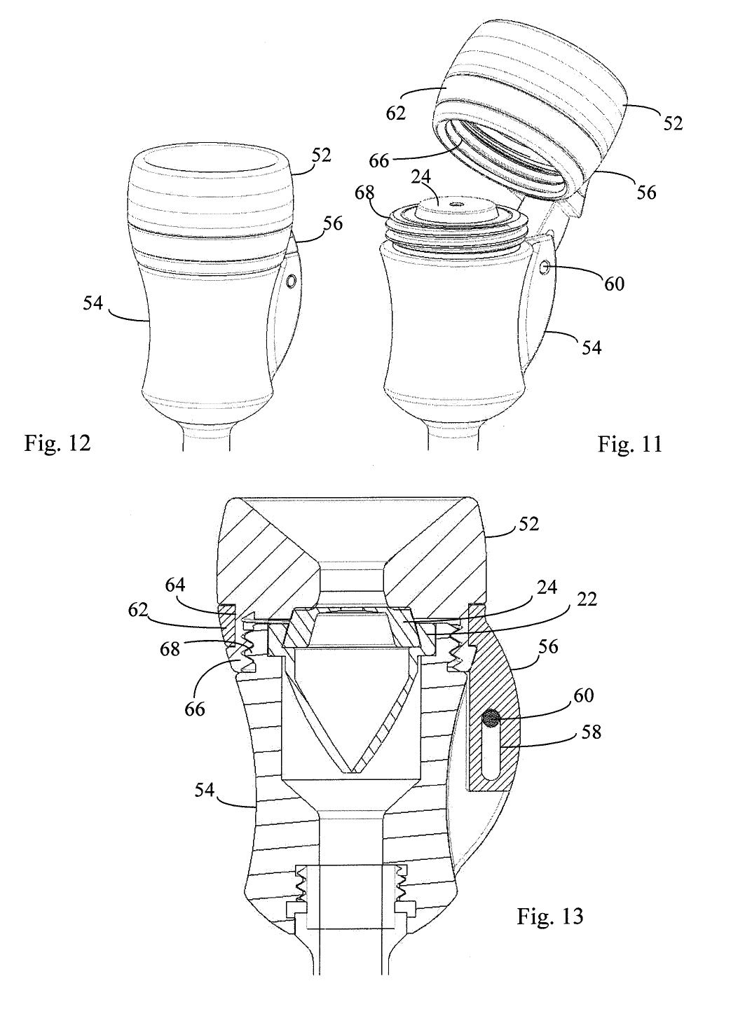

FIG. 11 depicts a perspective view of a cannula top according to a third embodiment, when the cannula top is in a first position for receiving a surgical seal;

FIG. 12 depicts a perspective view of the cannula top of FIG. 11, when the cannula top is in a second position enclosing a surgical seal;

FIG. 13 depicts a cross-section through the cannula top depicted in FIG. 12;

FIG. 14 depicts a perspective view of a cannula top according to a fourth embodiment, when the cannula top is in a first position for receiving a surgical seal;

FIG. 15 depicts a perspective view of the cannula top of FIG. 14, when the cannula top is in a second position enclosing a surgical seal;

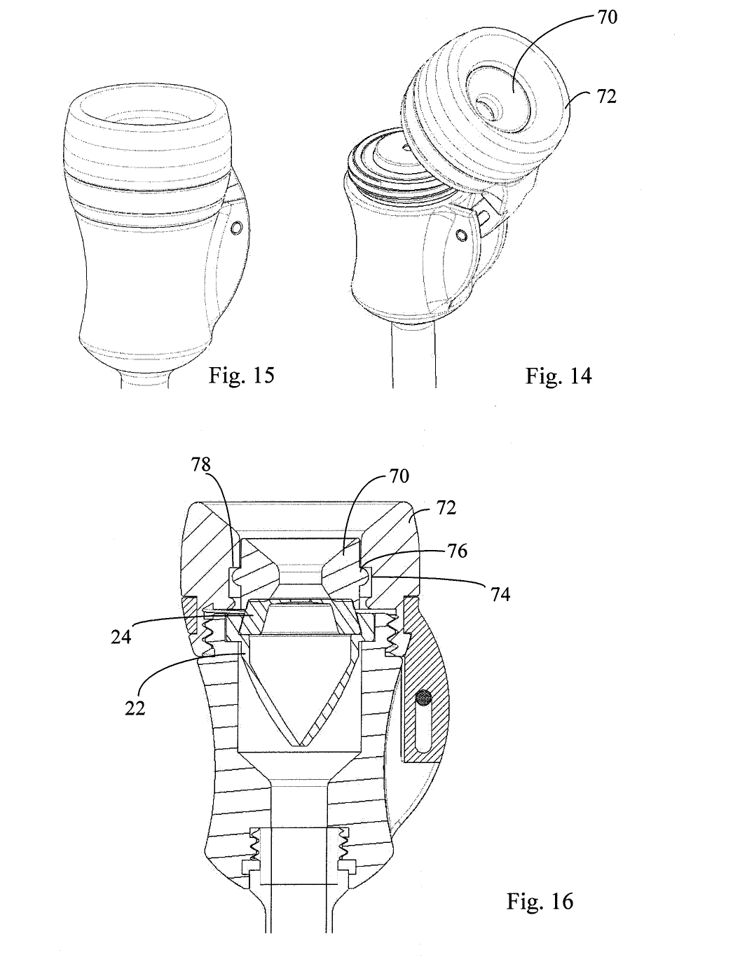

FIG. 16 depicts a cross-section through the cannula top depicted in FIG. 15;

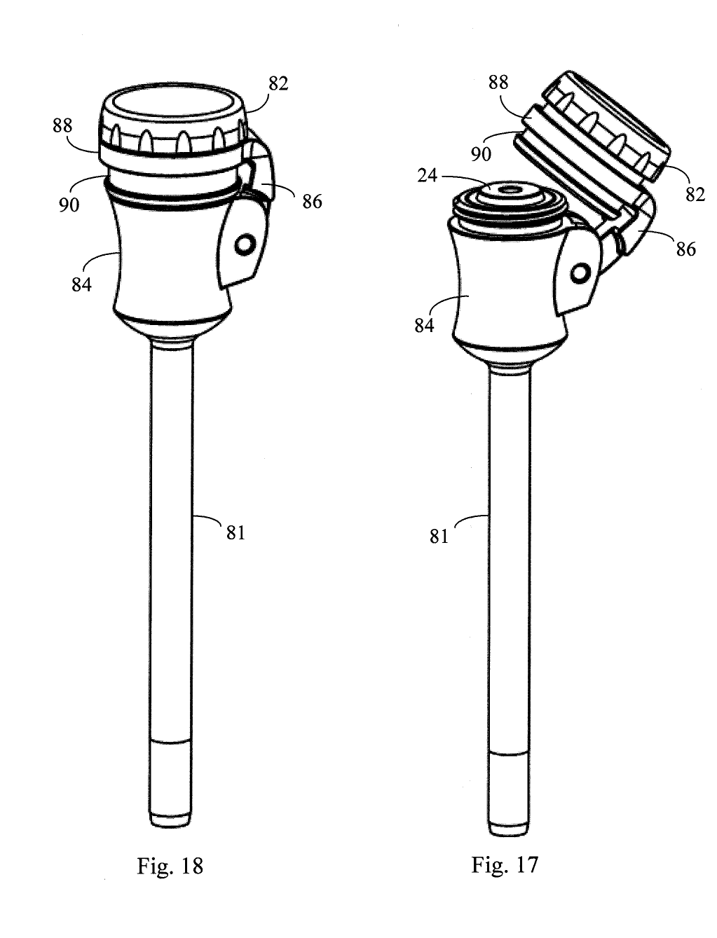

FIG. 17 depicts a perspective view of a cannula top according to a fifth embodiment, when the cannula top is in a first position for receiving a surgical seal;

FIG. 18 depicts a perspective view of the cannula top of FIG. 17, when the cannula top is in a second position enclosing a surgical seal;

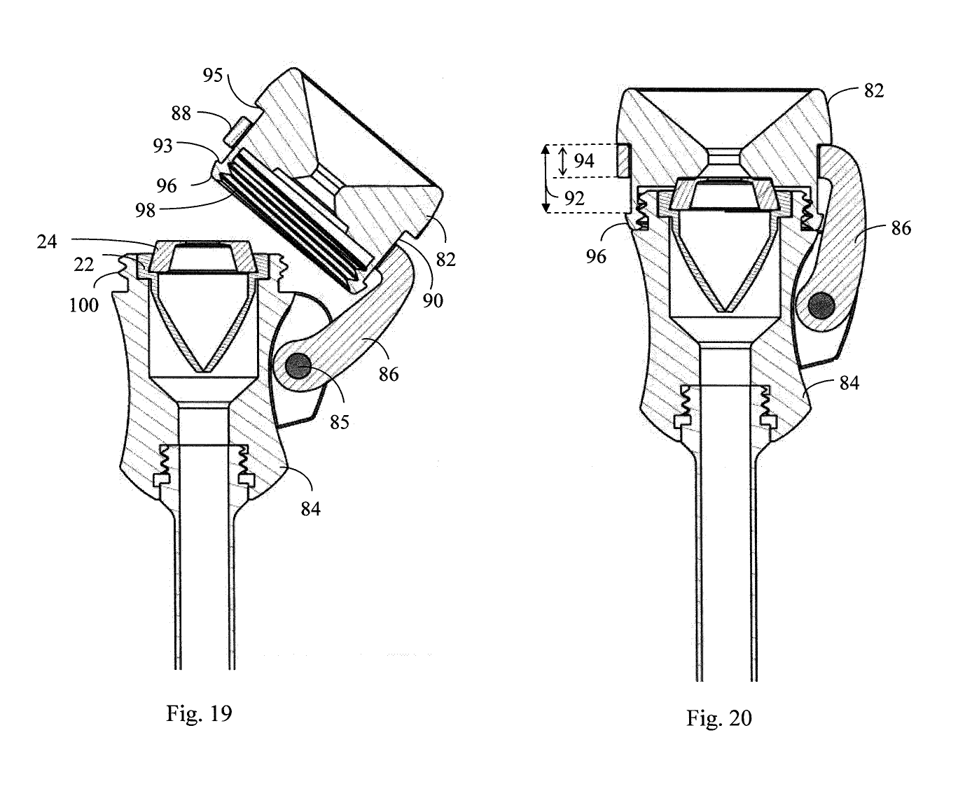

FIG. 19 depicts a cross section through the cannula top depicted in FIG. 17; and

FIG. 20 depicts a cross section through the cannula top depicted in FIG. 18.

FIG. 1 depicts a side view of a cannula top according to a first embodiment of the invention. The cannula top comprises a base member 2 to which a lid member 4 is attached. The base member 2 defines a longitudinal axis 6. A cannula 8 extends from the base member 2 of the cannula top in a distal direction, along the longitudinal axis 6. The lid member 4 is connected to the top of the base member 2 along the longitudinal axis 6. (Throughout this document, the distal direction refers to the direction along the longitudinal axis 6 away from the base member 2 towards the cannula 8. The proximal direction refers to the direction away from the cannula 8 towards the base member 2.)

The base member 2, lid member 4 and cannula 8 are preferably formed from a plastics material, for example Acetal (also known as polyoxymethylene or POM) or Polyether ether ketone (PEEK), although other materials may also be used.

The base member 2 has a through bore which is coaxial with the longitudinal axis and which defines a receptacle 10 for a surgical seal 12.

A pair of retaining arms 14 are provided on the base member 2 and positioned diametrically opposite each other about the longitudinal axis. The retaining arms 14 each comprise a projection 16 at their proximal end for engaging the lid member 4.

Each retaining arm 14 is formed from a single piece of resilient material, for example Acetal or PEEK although other materials may also be used. Each retaining arm 14 is pivotally attached to the base member 2 for rotation about an axis. A biasing element 18, which is a projection extending towards the longitudinal axis 6 in this embodiment, is provided on each retaining arm 14. Preferably the biasing element 18 is integrally formed with the retaining arm but in one embodiment (not illustrated) a biasing force may be provided by a separate resilient member, such as a spring or elastomer. The combination of biasing element 18 and the pivotal connection between the resilient arm 14 and the base member 2 act to position the projection 16 at a distance from the longitudinal axis 6 at which the projection 16 can engage a corresponding recess in the lid member 4. The retaining arm 14 may also be moved against the force applied by biasing element 18 by applying a force in the direction towards the longitudinal axis 6, so that the projection 16 moves towards the longitudinal axis.

FIG. 2 shows a side view in which resilient arm 14 on the left is in the biased position provided by the biasing element 18. Resilient arm 14 on the right in FIG. 2 is at the inward position, provided by applying an inward force to move the resilient arm 14 towards the longitudinal axis 6, against the force provided by the biasing element 18.

The base member 2 also comprises an optional luer connection 20. In alternate embodiments, luer connection 20 may be omitted.

The cannula 8 is releasably affixed to the base member 2 of the cannula top by a screw thread connection, most clearly seen in the exploded diagram of FIG. 3 and cross-section of FIG. 4. In alternate embodiments the cannula 8 may be integrally formed with the base member 2.

The surgical seal 12 is preferably formed from a relatively soft and flexible material, for example silicone. It comprises a lower seal 22 and an upper seal 24. The lower seal 22 comprises a generally wedge shaped element defining a deformable slit which is normally closed. Upper seal 24 has a frustoconical shape and defines a continuous rim around a deformable opening 26.

In use, when no instrument is inserted, the lower seal 22 provides an airtight seal. When a surgical instrument is inserted through the seal, it first passes through the opening 26 of upper seal 24. The diameter of the opening 26 is chosen to correspond to the diameter of the shaft of the surgical instrument, for example 3 mm, 5 mm or any other suitable size, so a seal is formed between the shaft of the surgical instrument and opening 26. As the instrument is inserted further, it then engages and opens the slit of lower seal 22. An air-tight seal is maintained the close fit of opening 26 on the shaft of the surgical instrument. When the instrument is removed, the slit of lower seal 22 closes before the instrument is removed from the opening 26 of upper seal 24. Thus, the seal maintains an air-tight opening to the body cavity throughout insertion and removal of a surgical instrument.

In order to retain the seal 12 within the cannula top so that an overall air-tight connection to the cannula 8 is achieved, the lid member 4 is movable between a first position and a second position. In the first position a seal 12 can be placed in the receptacle of the base member 2, for example as depicted in FIGS. 1, 2 and 4. The lid member 4 can then be moved into the second position where it engages the rim of the upper part 24 adjacent the opening 26 to provide an air-tight connection.

The lid member 4 is connected to the base member 2 by a track 28 extending from the lid member 4. The track is straight and engages a pin 30. The connection provided by the pin 30 in the track 28 enables the lid member 4 to be slid relative to the pin and rotated about the pin 30. This connection allows movement of the lid member 4 relative to the base member 2 with two degrees of freedom.

In use, the lid member 4 is translated into the second position by sliding the pin member 30 in the track 28 after aligning the lid member 4 so that its through bore is coaxial with the longitudinal axis 6. This enables the top part 24 of the seal to be engaged with a pure longitudinal force when the lid member is moved into the second position.

Lid member 4 defines recesses 32 which are engaged by projection 16 so that when the lid member 4 is in the second position relative to the base member 2, it is locked in position by a snap-fit connection between the projection 16 and the recess 32. FIGS. 5 and 6 depict two side views of the lid member 4 in the second position and FIG. 7 depicts a cross-section, in which the recess 32 can be seen most clearly.

When the lid member 4 is in the second position, it is retained in place by the interaction of the projection 16 of the retaining arms with the recess 32 of the lid member. As can be seen most clearly in FIG. 7, a portion of the lid member 4 engages the top portion 24 of the surgical seal. The bottom part 22 of the seal engages a portion of the base member 2. Together, these provide an airtight connection with the seal.

In order to remove the lid member 4 and enable removal of the seal 12, the retaining arms 16 are deformed inwardly against the force of biasing element 18. This releases the projection 16 from the recess 32 enabling translational movement of the lid member 4 away from the base member 2 along the longitudinal axis. The pivotal connection provided by the pin 30 within the track 28 then allows the lid to be rotated away from the longitudinal axis, allowing access to the seal.

A second embodiment of the invention is depicted in FIG. 8. FIG. 8 depicts a perspective view of a cannula top, an exploded view is depicted in FIG. 9, and a side view is depicted in FIG. 10. The construction of this second embodiment is the same as the first embodiment, except as described below, with the same parts using the same reference numerals.

In this embodiment a base member 42 is provided with retaining arms 44 which can be rotated outwardly from the longitudinal axis. Retaining arms 44 comprise a projection 46 at their proximal end. Retaining arms 44 are connected to base member 42 by a rotational connection at their distal end. Base member 42 defines a channel 48 for receiving a pin which passes through retaining arm 44. As depicted in FIG. 8, the retaining arm is normally positioned in a locking position. In this position, the projection 46 is positioned to engage a recess within the lid member 4 as described above for the first embodiment. The retaining arm 44 can also be rotated about connection 48 into an open position, in which the retaining arm 44 extends outwardly from the longitudinal axis. The open position allows improved access to the receptacle within the base member 42 for cleaning and also for removal of the surgical seal.

The perspective view in FIG. 8 also depicts how in this embodiment the optional luer port 50 is provided on the same side of the base member 42 as the connection to the lid member 4.

A third embodiment of the invention is depicted in FIGS. 11 to 13. The construction of this embodiment is the same as the first except as described below, and like reference numerals represent like parts.

FIG. 11 depicts a perspective view of a cannula top comprising a lid member 52 and a base member 54. The lid member 52 is connected to the base member 54 by a connecting member 56. The cannula top is shown in FIG. 11 in a first position, ready to receive a surgical seal. FIG. 12 depicts the cannula top in a second position, in which the surgical seal is enclosed within the cannula top. FIG. 13 depicts a cross section of the cannula top in the second position.

As can be seen most clearly in FIG. 13, the connecting member 56 defines a linear track 58 which is engaged by a pin 60 to connect the connecting member 56 to the base member 54. This connection provides two degrees of freedom with the base member 54, enabling the connecting member 56 to rotate about an axis which is defined by the pin 60 and also translate relative to the pin 60 along the track 58.

A rotatable connection is provided between the connecting member 56 and the lid member 52 by a hoop portion 62 of the connecting member 56 engaging a circular recess 64 defined by a surface of the lid member 52. A snap fit connection between the hoop portion 62 and circular recess 64 can provide attachment of the connecting member 56 to the lid member 52 while also allowing rotation.

The lid member 52 and base member 54 each comprise corresponding threads 66, 68, which are helical threads in this embodiment although any other suitable profile may be used. The thread 66 on the lid member 52 is formed on an internal surface and the thread 68 on the base member 54 is formed on an external surface.

In use, the lid member can be moved between a first position, depicted in FIG. 11, to a second position, depicted in FIGS. 12 and 13. In the first position, the pin 60 is located towards the distal end of the track 58 and the connecting member 56 is pivoted away from the base member 54 about the axis of the pin. This position allows access to a receptacle formed in the base member 54 for receiving a surgical seal. As in the above described embodiments, the surgical seal comprises a lower seal 22 and an upper seal 24.

In the second position, depicted in FIGS. 12 and 13, the surgical seal is retained within the receptacle of the base member 54 by the lid member 52. The lid member 52 is screwed into the second position by engaging the thread 66 with the thread 68. Hoop portion 62 of the connecting member 56 and recess 64 on the lid member 52 together enable the lid member 52 to rotate relative to the connecting member 56 to tighten and release the threaded connection between the lid member 52 and the base member 54. The linear track 58 allows the required translation of the lid member 52 along the axis of the threaded connection. As can be seen in FIG. 13, in the second position the pin 60 is located towards the proximal end of the track 58.

A fourth embodiment is depicted in FIGS. 14 to 16. The construction of this embodiment is the same as the third embodiment except as described below. In this embodiment a rotatable member 70 is provided which is contained within the lid member 72 and free to rotate and translate relative to the lid member 72.

The lid member 72 defines an internal circular recess 74 which engages a protrusion 76 provided on the rotatable member 70. Preferably, protrusion 76 defines a continuous ring around the rotatable member 70. The internal circular recess 74 has a depth in the direction along the axis of the thread which is greater than the depth of the protrusion 76. Thus, the recess allows rotation and translation of the rotatable member relative to the lid member 72.

In use, when the lid member 72 is moved into the second position by engaging the threads, the rotatable member prevents the transfer of rotation shear forces from the lid member 72 to the upper part 24 of the surgical seal. The internal circular recess 74 comprises a proximal face 78 which is positioned to engages the protrusion 76 in the first position. This ensures that a longitudinal force is transferred from the lid member 72 through the rotatable member 70 to engage the upper part 24 of the surgical seal.

A fifth embodiment of the invention is depicted in FIGS. 17 to 20. The construction of this embodiment is the same as the first except as described below. This embodiment is currently the most preferred. It is similar to the third embodiment but differs in the way in which the lid member 82 is connected to the base member 84.

FIG. 17 depicts a perspective view of a cannula top comprising a lid member 82 and a base member 84, which is provided on top of a cannula 81. The lid member 82 is connected to the base member 84 by a connecting member 86. The cannula top is shown in FIG. 17 in a first position, ready to receive a surgical seal. FIG. 18 depicts the cannula top in a second position, in which the surgical seal is enclosed within the cannula top. FIG. 19 depicts a cross section of the cannula top in the first position and FIG. 20 depicts a cross sections of the cannula top in the second position.

A rotatable or pivotal connection is provided between the connecting member 86 and the base member 84 by a pin joint 85. A rotatable and translatable connection is provided between the connecting member 86 and the lid member 82 by a hoop or ring portion 88 of the connecting member 86 which engages a circular recess or cylindrical portion 90 defined by a surface of the lid member 82. A first shoulder 93 and a second shoulder 95 at the interface to a larger diameter portion of the lid member 82 define the length 92 of the cylindrical portion 90. The length 92 is greater than the axial thickness 94 of the ring portion 88. Thus, the lid member 82 can both translate and rotate relative to the ring portion 88 providing two degrees of freedom in the connection between the lid member 82 and the connecting member 86. The ring portion 88 is preferably snap fit onto the cylindrical portion 88 by passing it over a tapered end 96 of the lid member 82 adjacent the first shoulder 93. This construction can provide a more robust joint which is easier to clean than the pin and track connection used in the embodiment of FIG. 11.

The lid member 82 and base member 84 each comprise corresponding threads 98, 100, which are helical threads in this embodiment although any other suitable profile may be used. The thread 98 on the lid member 82 is formed on an internal surface and the thread 100 on the base member 84 is formed on an external surface.

In use, the lid member 82 can be moved between a first position, depicted in FIGS. 17 and 19, and a second position, depicted in FIGS. 18 and 20. In the first position, the ring portion 88 is located towards the first shoulder 93 and the connecting member 86 is pivoted away from the base member 84 about the axis of the pin 85. This position allows access to a receptacle formed in the base member 84 for receiving a surgical seal. As in the above described embodiments, the surgical seal comprises a lower seal 22 and an upper seal 24.

In the second position, depicted in FIGS. 18 and 20, the surgical seal is retained within the receptacle of the base member 84 by the lid member 82. The lid member 82 is screwed into the second position by engaging the thread 98 with the thread 100. Ring portion 88 of the connecting member 86 and cylindrical portion 90 on the lid member 82 together enable the lid member 82 to rotate relative to the connecting member 86 to tighten and release the threaded connection between the lid member 82 and the base member 84. During tightening and release of the threaded connection, the ring portion 88 can translate along the cylindrical portion 90, providing the required translation of the lid member 82 along the axis of the threaded connection. As can be seen in FIGS. 18 and 20, in the second position the ring portion 88 is located closer to the second shoulder 95 than in the first position.

The features of the embodiments may be described in ways other than those explicitly described. For example, the embodiment of FIG. 17 may include an additional rotatable member as described in the embodiment of FIG. 14.

The present invention therefore provides a cannula top in which the lid of the cannula top remains connected to the base, ensuring that the lid and base cannot easily be separated in the operating theatre or during sterilisation/decontamination. Some embodiments engage a surgical seal purely in a longitudinal direction, minimising the effect of any shear forces acting on the surgical seal.

1. A cannula top for enclosing a surgical seal, wherein the cannula top comprises:
a base member having a through bore which defines a receptacle for the surgical seal;
a lid member having a through bore, wherein the lid member is movable between a first position in which the surgical seal can be inserted into and removed from the receptacle, and a second position in which the surgical seal is engaged by the lid member and retained in the receptacle by the lid member; and
a connecting member which connects the lid member to the base member in both the first position and the second position.

2. A cannula top according to claim 1, wherein the base member and the lid member each define respective threads and the lid member is retained in the second position by the engagement of the thread of the base member with the thread of the lid member.

3. A cannula top according to claim 1, wherein the lid member is rotatably connected to the connecting member for rotation relative to the connecting member about the axis of the through bore of the lid member.

4. A cannula top according to claim 1, wherein the lid member is connected to the connecting member for translation relative to the connecting member in the direction of the axis of the through bore defined by the lid member.

5. A cannula top according to claim 1, wherein the lid member defines an outer surface which comprises a cylindrical portion and the connecting member engages the lid member via a ring which surrounds the cylindrical portion.

6. A cannula top according to claim 5, where in the cylindrical portion has a length which is greater than the axial thickness of the ring.

7. A cannula top according to claim 1, wherein the connecting member is pivotally connected to the base member.

8. A cannula top according to claim 1, further comprising attachment means for releasable attachment to an end of a cannula

9. A system comprising:
a cannula top according to claim 1; and
a surgical seal.

10. A cannula top according to claim 2, wherein the lid member is rotatably connected to the connecting member for rotation relative to the connecting member about the axis of the through bore of the lid member.

11. A cannula top according to claim 10, wherein the lid member is connected to the connecting member for translation relative to the connecting member in the direction of the axis of the through bore defined by the lid member.

12. A cannula top according to claim 11, wherein the lid member defines an outer surface which comprises a cylindrical portion and the connecting member engages the lid member via a ring which surrounds the cylindrical portion.

13. A cannula top according to claim 12, where in the cylindrical portion has a length which is greater than the axial thickness of the ring.

14. A cannula top according to claim 13, wherein the connecting member is pivotally connected to the base member.

15. A cannula top according to claim 14, further comprising attachment means for releasable attachment to an end of a cannula
